(19) United States
(12) Patent Application Publication
(10) Pub. No.: US 2022/0032029 A1
(43) Pub. Date: Feb. 3, 2022

(54) MECHANISMS FOR PREVENTING RECONNECTION OF CONNECTORS INTENDED FOR SINGLE USE

(71) Applicant: BAYER HEALTHCARE LLC, WHIPPANY, NJ (US)

(72) Inventors: CHRISTOPHER LAZZARA, CRANBERRY TWP., PA (US); MICHAEL SWANTNER, SAXONBURG, PA (US); DANIEL MOORE, III, PITTSBURGH, PA (US); RONALD HELLER, MONROEVILLE, PA (US); BARRY TUCKER, VERONA, PA (US)

(21) Appl. No.: 17/413,607

(22) PCT Filed: Dec. 18, 2019
(86) PCT No.: PCT/US2019/067089

§ 371 Date: Jun. 14, 2021

Related U.S. Application Data
(60) Provisional application No. 62/782,695, filed on Dec. 20, 2018.

(51) Int. Cl. A61M 39/10

(52) U.S. Cl. CPC A61M 39/1011; A61M 2205/276; A61M 2205/273

(57) ABSTRACT

A single-use connector configured to be removably connected to a reusable connector includes a hollow, tubular housing having an open proximal side opposite a distal side along a longitudinal length of the housing and at least one guard member connected to the tubular housing. The at least one guard member is configured to transition between an open position, in which the single-use connector can be connected to the reusable connector, and a closed position in which the guard member prevents the single-use connector from being connected to the reusable connector. Disconnecting the single-use connector from the reusable connector causes the at least one guard member to transition from the open position to the closed position. A single-use disposable set (SUDS) including a single-use connector and at least use guard member for preventing reconnection of the connector to a reusable connector or fluid port is also disclosed.

### CROSS-REFERENCE TO RELATED APPLICATION

This application claims priority to U.S. Provisional Application No. 62/782,695, filed on Dec. 20, 2019, the disclosure of which is hereby incorporated by reference in its entirety.

### BACKGROUND

### Field

This invention relates, in general, to the field of medical connectors and, more particularly, to assemblies including a single-use connector configured to prevent the single-use connector from being reconnected to a reusable connector.

### Description of the Related Art

Typical medical connectors are provided for use with various medical devices, including dosage containers, administration sets, catheters, medical lines, fluid delivery devices, and automated fluid injectors. These connectors are commonly used in medical procedures where it is necessary to, for example, draw a drug from a container or to deliver medical fluid(s) to one or more patients. Since such connectors can come into contact with patient fluids, the connectors are often single-use connectors that are discarded after each use.

Various conventional two-piece, single-use medical connectors for establishing fluid communication between, for example, an injection device and a patient line are known. Exemplary two-piece medical connectors are disclosed in U.S. Pat. No. 4,981,469 to Whitehouse et al. (hereinafter “the Whitehouse patent”) and U.S. Pat. No. 7,241,285 to Dikeman (hereinafter “the Dikeman patent”). The Whitehouse patent is directed to an assembly for connection to a medicant supply tube and includes a septum assembly having a distal end cap, a septum cap, and an external adapter. The Dikeman patent discloses a connector for connecting a fluid passage device, such as may be provided by a Luer taper, and an injection site having a thin diaphragm. The connector includes a reduced diameter cannula for engaging the thin diaphragm sufficiently to open the thin diaphragm to establish an open fluid passage. U.S. Pat. No. 6,911,025 to Miyahara is directed to a dialysis connector in which a patient side connector is enclosed by a long term use protective cap which encloses a sterile inner cap that is replaced at the conclusion of each dialysis procedure.

Such conventional two-piece medical connectors can be disconnected and reconnected multiple times. Further, there is often no way to visually determine whether the conventional medical connectors have been used previously. Accordingly, users may inadvertently or intentionally disconnect and reconnect a medical connector, which should be discarded after a single use. Users may also accidently connect a previously used medical connector to a fluid set because they cannot tell that the connector has already been used.

### SUMMARY

In view of the foregoing, a need exists for single-use connectors having features for preventing accidental or intentional reconnection and/or reuse. The connectors and medical connector assemblies disclosed herein include features for preventing users from reconnecting the single-use connector to other portions of a fluid path set or fluid delivery device. Disclosed herein is a plurality of such reconnection prevention mechanisms.

According to some non-limiting embodiments or aspects of the present disclosure, a single-use connector configured to be removably connected to a reusable connector may include a hollow, tubular housing having an open proximal side opposite a distal side along a longitudinal length of the housing and at least one guard member connected to the tubular housing. The at least one guard member may be configured to transition between an open position in which the single-use connector can be connected to the reusable connector and a closed position in which the at least one guard member prevents the single-use connector from being connected to the reusable connector. Disconnecting the single-use connector from the reusable connector may cause the at least one guard member to transition from the open position to the closed position.

According to some non-limiting embodiments or aspects of the present disclosure, at least one thread-less attachment member may be provided and configured to be connected to a thread-less attachment member of the reusable connector to establish a fluid connection between the single-use connector and the reusable connector. The at least one thread-less attachment member of the single-use connector may have at least one of a male luer connector and a female luer connector. An O-ring may be provided around at least a portion of the housing for sealing the single-use connector to the reusable connector.

According to some non-limiting embodiments or aspects of the present disclosure, the at least one guard member may have an annular compression sleeve at least partially enclosing the open proximal end of the housing defining an interior and a proximal opening, wherein the distal end of the reusable connector can be inserted into the interior of the compression sleeve through the proximal opening and connected to the proximal end of the single-use connector when the at least one guard member is in the open position, and wherein, when the connectors are attached together, the at least one guard member engages at least a portion of the reusable connector to reinforce the connection between the single-use connector and the reusable connector. The compression sleeve may have an elastomeric sleeve biased to the closed position, and wherein, when in the closed position, the sleeve at least partially covers the open proximal end of the housing to prevent reconnection of the single-use connector to the reusable connector. Disconnecting the single-use connector from the reusable connector may reduce a diameter of the opening defined by the compression sleeve, thereby preventing the single-use connector from being reconnected to the reusable connector. The compression sleeve is biased to the closed position. The compression sleeve may have a distal portion mounted to and extending proximally and radially outwardly from the housing of the single-use connector, and a proximal portion connected to and extending proximally and radially inwardly from the distal portion of the compression sleeve, wherein the proximal portion of the compression sleeve defines the opening of the sleeve.

According to some non-limiting embodiments or aspects of the present disclosure, the at least one guard member may have at least a first arm and a second arm, wherein the first arm and the second arm each may have a first portion connected to and extending proximally and radially outwardly from the housing of the single-use connector and a second portion connected to and extending proximally and radially inwardly from the first portion of the arm. When the single-use connector is connected to the reusable connector, an inwardly directed surface of the second portion of each arm may engage a portion of the reusable connector to reinforce a connection between the single-use connector and the reusable connector. Disconnecting the single-use connector from the reusable connector may cause the arms to pivot radially inwardly relative to the housing of the single-use connector, such that an outer surface of the second portion of the arms blocks reconnection of the single-use connector with the reusable connector.

According to some non-limiting embodiments or aspects of the present disclosure, the at least one guard member may be slidably positioned in the housing and wherein, when the single-use connector is in the closed position, a portion of the at least one guard member extends from the open proximal end of the housing to prevent the single-use connector from being reconnected to the reusable connector. The at least one guard member may have a spring configured to bias the guard member to the closed position, a deployable support configured to transition from a retracted position to a deployed position, and at least one rod extending between the spring and the deployable support. A maximum outer diameter of the deployable support may be greater when in the deployed position than when in the retracted position. The deployable support may be maintained in the retracted position by a narrowed inner surface of a sidewall of the housing. The deployable support may transition to the deployed position once it extends beyond the open proximal end of the housing.

According to some non-limiting embodiments or aspects of the present disclosure, a single-use medical connector may include a housing configured to be inserted through a corresponding slot of a fluid injector for connection to a multi-use disposable set installed therein, a fluid inlet port extending through the housing configured for removable engagement with a fluid connection port of the multi-use disposable set of the injector to establish a fluid connection therewith, a waste outlet port extending through the housing configured for removable engagement with a waste inlet port of the multi-use disposable set to establish a fluid connection therewith, and a patient fluid line providing a fluid connection between the fluid inlet port and the waste outlet port. The patient fluid line may have a fluid port connector connected to the fluid inlet port, an end port connector connected to the waste outlet port, and flexible tubing extending between the fluid port connector and the end port connector. The end port connector may be configured to be disconnected from the waste outlet port so that fluid can be provided from the fluid inlet port via the patient line to a patient while the waste outlet port remains engaged with the waste inlet port of the multi-use disposable set. A reconnection prevention mechanism may be configured to prevent reconnection of the fluid inlet port with the fluid connection port of the multi-use disposable set after the fluid inlet port is disconnected from the fluid connection port.

According to some non-limiting embodiments or aspects of the present disclosure, the reconnection prevention mechanism may have at least one guard member disposed on the fluid inlet port. The at least one guard member may be configured to engage the connection port when the single-use medical connector is connected to the multi-use disposable set and, upon disconnecting the fluid inlet from the connection port, the at least one guard member may be configured to transition to an extended position in which the at least one guard member blocks at least a portion of the fluid inlet port to prevent the fluid inlet port from being reconnected to the connection port.

According to some non-limiting embodiments or aspects of the present disclosure, the housing of the single-use connector may have a latch mechanism, the latch mechanism being configured to prevent the fluid inlet port from being reconnected to the fluid connection port of the multi-use disposable set after the fluid inlet port has once been engaged with and subsequently disengaged from the fluid connection port. The latch mechanism may have a flexible beam which is depressed by a user to disconnect the housing from the multi-use disposable set and a finger catch that engages the housing when the flexible beam is depressed, thereby maintaining the latch mechanism in a blocking position in which the fluid inlet port is prevented from being reconnected to the fluid connection port.

According to some non-limiting embodiments or aspects of the present disclosure, the fluid inlet port may have a deflectable flange that is movable between a first, retracted position configured to permit connection of the fluid inlet port with the fluid connection port of the multi-use disposable set, and a second, extended position configured to prevent connection of the fluid inlet port with the fluid connection port of the multi-use disposable set. The deflectable flange may have a distally facing angled surface angled such that the fluid inlet port can be removed from the fluid connection port and a flat proximal surface angled to block the fluid inlet port from reconnecting with the fluid connection port.

According to some non-limiting embodiments or aspects of the present disclosure, a single-use disposable set for use in administering fluid to a patient may have a housing configured to be inserted through a corresponding slot defined within an enclosure of a fluid injector. Upon installation of a multi-use disposable set within the fluid injector, a fluid connection port and a waste inlet port of the multi-use disposable set may be accessible within the corresponding slot of the fluid injector. The single-use disposable set may further have a fluid inlet port defined through the housing, the fluid inlet port having a proximal side thereof being configured for connection to the fluid connection port of the multi-use disposable set. The single-use disposable set may further have a waste outlet port defined through the housing, the waste outlet port having a proximal side being configured for connection to the waste inlet port of the multi-use disposable set. The single-use disposable set may further have a patient fluid line having a fluid port connector, an end port connector and a flexible tubing extending between the fluid port connector and the end port connector. The fluid port connector may be connected to a distal side of the fluid inlet port and the end port connector being removably connectable to a distal side of the waste outlet port such that (A) upon insertion of the housing into the corresponding slot of the fluid injector, the fluid inlet port and the waste outlet port connect, respectively, to the fluid connection port and the waste inlet port thereby enabling fluid communication between the multi-use disposable set and the single-use disposable set, and (B) upon removal of the housing from the corresponding slot, the fluid inlet port and the waste outlet port of the single-use disposable set disconnect, respectively, from the fluid connection port and the waste inlet port of the multi-use disposable set; and a reconnection prevention mechanism that, upon removal of the housing from the corresponding slot, is configured to prevent reconnection of at least one of (A) the fluid inlet port of the single-use disposable set to the fluid connection port of the multi-use disposable set and (2) the waste outlet port of the single-use disposable set to the waste inlet port of the multi-use disposable set.

According to some non-limiting embodiments or aspects of the present disclosure, a method for providing a sterile connection between a single use connector and a reusable connector may be provided. The method may include providing at least one reusable connector having a hollow, tubular housing having a proximal side opposite a distal side along a longitudinal length of the housing. The method further may include providing at least one single-use connector having a hollow, tubular housing having a proximal side opposite a distal side along a longitudinal length of the housing and at least one guard member connected to the tubular housing configured to transition between an open position in which the single-use connector can be connected to the reusable connector and a closed position in which the guard member prevents the single-use connector from being connected to the reusable connector. The method further may include coupling the reusable connector to the single-use connector by aligning the distal side of the reusable connector for releasably connecting to the proximal side of a first single-use connector. The method further may include disconnecting the single-use connector from the reusable connector, thereby causing the at least one guard member of the single-use connector to transition to the closed position to prevent reuse of the single-use connector. The at least one guard member may have a compression sleeve. Coupling the reusable connector to the single-use connector may cause the compression sleeve to engage at least a portion of the hollow, tubular housing of the reusable connector to reinforce the connection between the single-use connector and the reusable connector.

Further aspects of the single-use connector assembly and method are disclosed in the following enumerated clauses.

Clause 1: A single-use connector configured to be removably connected to a reusable connector, the single-use connector comprising: a hollow, tubular housing having an open proximal side opposite a distal side along a longitudinal length of the housing; and at least one guard member connected to the tubular housing configured to transition between an open position in which the single-use connector can be connected to the reusable connector and a closed position in which the at least one guard member prevents the single-use connector from being connected to the reusable connector, wherein disconnecting the single-use connector from the reusable connector causes the at least one guard member to transition from the open position to the closed position.

Clause 2: The single-use connector of clause 1, further comprising at least one thread-less attachment member configured to be connected to a thread-less attachment member of the reusable connector to establish a fluid connection between the single-use connector and the reusable connector.

Clause 3: The single-use connector of clause 2, wherein the at least one thread-less attachment member of the single-use connector comprises at least one of a male luer connector and a female luer connector.

Clause 4: The single-use connector of any of clauses 1-3, further comprising an O-ring provided around at least a portion of the housing for sealing the single-use connector to the reusable connector.

Clause 5: The single-use connector of any of clauses 1-4, wherein the at least one guard member comprises an annular compression sleeve at least partially enclosing the open proximal end of the housing defining an interior and a proximal opening, wherein the distal end of the reusable connector can be inserted into the interior of the compression sleeve through the proximal opening and connected to the proximal end of the single-use connector when the at least one guard member is in the open position, and wherein, when the connectors are attached together, the at least one guard member engages at least a portion of the reusable connector to reinforce the connection between the single-use connector and the reusable connector.

Clause 6: The single-use connector of clause 5, wherein the compression sleeve comprises an elastomeric sleeve biased to the closed position, and wherein, when in the closed position, the sleeve at least partially covers the open proximal end of the housing to prevent reconnection of the single-use connector to the reusable connector.

Clause 7: The single-use connector of clause 5 or clause 6, wherein disconnecting the single-use connector from the reusable connector reduces a diameter of the opening defined by the compression sleeve, thereby preventing the single-use connector from being reconnected to the reusable connector.

Clause 8: The single-use connector of any of clauses 5-7, wherein the compression sleeve is biased to the closed position.

Clause 9: The single-use connector of any of clauses 5-8, wherein the compression sleeve comprises a distal portion mounted to and extending proximally and radially outwardly from the housing of the single-use connector, and a proximal portion connected to and extending proximally and radially inwardly from the distal portion of the compression sleeve, wherein the proximal portion of the compression sleeve defines the opening of the sleeve.

Clause 10: The single-use connector of any of clauses 1-4, wherein the at least one guard member comprises at least a first arm and a second arm, wherein the first arm and the second arm each comprise a first portion connected to and extending proximally and radially outwardly from the housing of the single-use connector and a second portion connected to and extending proximally and radially inwardly from the first portion of the arm.

Clause 11: The single-use connector of clause 10, wherein, when the single-use connector is connected to the reusable connector, an inwardly directed surface of the second portion of each arm engages a portion of the reusable connector to reinforce a connection between the single-use connector and the reusable connector.

Clause 12: The single-use connector of clause 10 or clause 11, wherein disconnecting the single-use connector from the reusable connector causes the arms to pivot radially inwardly relative to the housing of the single-use connector, such that an outer surface of the second portion of the arms blocks reconnection of the single-use connector with the reusable connector.

Clause 13: The single-use connector of any of clauses 1-4, wherein the at least one guard member is slidably positioned in the housing and wherein, when the single-use connector is in the closed position, a portion of the at least one guard member extends from the open proximal end of the housing to prevent the single-use connector from being reconnected to the reusable connector.

Clause 14: The single-use connector of clause 13, wherein the at least one guard member comprises a spring configured to bias the guard member to the closed position, a deployable support configured to transition from a retracted position to a deployed position, and at least one rod extending between the spring and the deployable support, and wherein a maximum outer diameter of the deployable support is greater when in the deployed position than when in the retracted position.

Clause 15: The single-use connector of clause 14, wherein the deployable support is maintained in the retracted position by a narrowed inner surface of a sidewall of the housing, and wherein the deployable support transitions to the deployed position once it extends beyond the open proximal end of the housing.

Clause 16: A single-use medical connector comprising: a housing configured to be inserted through a corresponding slot of a fluid injector for connection to a multi-use disposable set installed therein; a fluid inlet port extending through the housing configured for removable engagement with a fluid connection port of the multi-use disposable set of the injector to establish a fluid connection therewith; a waste outlet port extending through the housing configured for removable engagement with a waste inlet port of the multi-use disposable set to establish a fluid connection therewith; and a patient fluid line providing a fluid connection between the fluid inlet port and the waste outlet port, the patient fluid line comprising a fluid port connector connected to the fluid inlet port, an end port connector connected to the waste outlet port, and flexible tubing extending between the fluid port connector and the end port connector, wherein the end port connector is configured to be disconnected from the waste outlet port so that fluid can be provided from the fluid inlet port via the patient line to a patient while the waste outlet port remains engaged with the waste inlet port of the multi-use disposable set, and wherein a reconnection prevention mechanism is configured to prevent reconnection of the fluid inlet port with the fluid connection port of the multi-use disposable set after the fluid inlet port is disconnected from the fluid connection port.

Clause 17: The single-use medical connector of clause 16, wherein the reconnection prevention mechanism comprises at least one guard member disposed on the fluid inlet port, the at least one guard member being configured to engage the connection port when the single-use medical connector is connected to the multi-use disposable set and, upon disconnecting the fluid inlet from the connection port, the at least one guard member is configured to transition to an extended position in which the at least one guard member blocks at least a portion of the fluid inlet port to prevent the fluid inlet port from being reconnected to the connection port.

Clause 18: The single-use medical connector of clause 16 or clause 17, wherein the housing of the single-use connector comprises a latch mechanism, the latch mechanism being configured to prevent the fluid inlet port from being reconnected to the fluid connection port of the multi-use disposable set after the fluid inlet port has once been engaged with and subsequently disengaged from the fluid connection port.

Clause 19: The single-use medical connector of clause 18, wherein the latch mechanism comprises a flexible beam which is depressed by a user to disconnect the housing from the multi-use disposable set and a finger catch that engages the housing when the flexible beam is depressed, thereby maintaining the latch mechanism in a blocking position in which the fluid inlet port is prevented from being reconnected to the fluid connection port.

Clause 20: The single-use medical connector of any of clauses 16-19, wherein the fluid inlet port comprises a deflectable flange that is movable between a first, retracted position configured to permit connection of the fluid inlet port with the fluid connection port of the multi-use disposable set, and a second, extended position configured to prevent connection of the fluid inlet port with the fluid connection port of the multi-use disposable set.

Clause 21: The single-use medical connector of clause 20, wherein the deflectable flange comprises a distally facing angled surface angled such that the fluid inlet port can be removed from the fluid connection port and a flat proximal surface angled to block the fluid inlet port from reconnecting with the fluid connection port.

Clause 22: A single-use disposable set for use in administering fluid to a patient, the single-use disposable set comprising: a housing configured to be inserted through a corresponding slot defined within an enclosure of a fluid injector wherein, upon installation of a multi-use disposable set within the fluid injector, a fluid connection port and a waste inlet port of the multi-use disposable set are accessible within the corresponding slot of the fluid injector; a fluid inlet port defined through the housing, the fluid inlet port having a proximal side thereof being configured for connection to the fluid connection port of the multi-use disposable set; a waste outlet port defined through the housing, the waste outlet port having a proximal side being configured for connection to the waste inlet port of the multi-use disposable set; a patient fluid line having a fluid port connector, an end port connector and a flexible tubing extending between the fluid port connector and the end port connector, the fluid port connector being connected to a distal side of the fluid inlet port and the end port connector being removably connectable to a distal side of the waste outlet port; such that (A) upon insertion of the housing into the corresponding slot of the fluid injector, the fluid inlet port and the waste outlet port connect, respectively, to the fluid connection port and the waste inlet port thereby enabling fluid communication between the multi-use disposable set and the single-use disposable set, and (B) upon removal of the housing from the corresponding slot, the fluid inlet port and the waste outlet port of the single-use disposable set disconnect, respectively, from the fluid connection port and the waste inlet port of the multi-use disposable set; and a reconnection prevention mechanism that, upon removal of the housing from the corresponding slot, is configured to prevent reconnection of at least one of (A) the fluid inlet port of the single-use disposable set to the fluid connection port of the multi-use disposable set and (2) the waste outlet port of the single-use disposable set to the waste inlet port of the multi-use disposable set.

Clause 23: A method for providing a sterile connection between a single use connector and a reusable connector, the method comprising the steps of: providing at least one reusable connector comprising a hollow, tubular housing having a proximal side opposite a distal side along a longitudinal length of the housing; providing at least one single-use connector comprising a hollow, tubular housing having a proximal side opposite a distal side along a longitudinal length of the housing and at least one guard member connected to the tubular housing configured to transition between an open position in which the single-use connector can be connected to the reusable connector and a closed position in which the guard member prevents the single-use connector from being connected to the reusable connector; coupling the reusable connector to the single-use connector by aligning the distal side of the reusable connector for releasably connecting to the proximal side of a first single-use connector; and disconnecting the single-use connector from the reusable connector, thereby causing the at least one guard member of the single-use connector to transition to the closed position to prevent reuse of the single-use connector.

Clause 24: The method of clause 23, wherein the at least one guard member comprises a compression sleeve, and wherein coupling the reusable connector to the single-use connector causes the compression sleeve to engage at least a portion of the hollow, tubular housing of the reusable connector to reinforce the connection between the single-use connector and the reusable connector.

These and other features and characteristics of single-use disposable set connectors, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure. As used in the specification and the claims, the singular form of “a”, “an”, and “the” include plural referents unless the context clearly dictates otherwise.

### BRIEF DESCRIPTION OF THE DRAWINGS

FIG. 1A is a cross-sectional view of a medical connector in a pre-use or open position according to some non-limiting embodiments or aspects of the disclosure;

FIG. 1B is a cross-sectional view of the medical connector of FIG. 1A in a connected or in-use position;

FIG. 1C is cross-sectional view of the medical connector of FIG. 1A in a post-use or closed position;

FIG. 2A is a cross-sectional view of another embodiment of a medical connector in a pre-use or open position according to some non-limiting embodiments or aspects of the disclosure;

FIG. 2B is cross-sectional view of the medical connector of FIG. 2A in a connected or in-use position;

FIG. 2C is cross-sectional view of the medical connector of FIG. 2A in a post-use or closed position;

FIG. 3A is a cross-sectional view of another embodiment of a medical connector in a connected or in-use position according to some non-limiting embodiments or aspects of the disclosure;

FIG. 3B is cross-sectional view of the medical connector of FIG. 3A in a post-use or closed position;

FIG. 4A is a cross-sectional view of a medical connector in a pre-use or open position according to some non-limiting embodiments or aspects of the disclosure;

FIG. 4B is cross-sectional view of the medical connector of FIG. 4A in a connected or in-use position;

FIG. 4C is cross-sectional view of the medical connector of FIG. 4A in a post-use or closed position;

FIG. 5A is a perspective view of a prior art fluid injector system into which a prior art multi-use disposable set (MUDS) has been installed;

FIG. 5B is a perspective view of a portion of the fluid injector system of FIG. 5A showing a prior art single-use disposable set (SUDS) prior to its insertion through an opening or slot of the fluid injection system and into the MUDS installed therein;

FIG. 5C is a perspective view of the prior art SUDS of FIG. 5B;

FIG. 6 is a perspective view of an embodiment of a single-use disposable set (SUDS) modified to include a reconnection prevention mechanism according to some non-limiting embodiments or aspects of the disclosure;

FIG. 7A is a schematic cross-sectional view of another embodiment of a SUDS in a connected position according to some non-limiting embodiments or aspects of the disclosure;

FIG. 7B is schematic cross-sectional view of the SUDS of FIG. 7A in a disconnected position;

FIG. 8A is a schematic cross-sectional view of another embodiment of a SUDS in a connected position according to some non-limiting embodiments or aspects of the disclosure;

FIG. 8B is schematic cross-sectional view of the SUDS of FIG. 8A in a disconnected position;

FIG. 9A is a schematic cross-sectional view of a portion of the SUDS of FIG. 6 prior to insertion into a MUDS;

FIG. 9B is a cross-sectional view of a portion of the SUDS of FIG. 6 while it is inserted into the MUDS; and

FIG. 9C is schematic cross-sectional view of a portion of the SUDS of FIG. 6 after being removed from a MUDS.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to a medical connector including a reusable connector connected at one end to a fluid source and at an opposite end to a single use connector, the term “proximal” refers to portions of the connectors closest to the fluid source. The term “distal” refers to the portions of the connectors farthest from the fluid source. For example, for a single-use connector between a reusable connector of a fluid injector and patient catheter, a “proximal side” of the single-use connector refers to the portion of the connector connected to the reusable connector. The “distal side” of the connector refers to the portion of the connector closest to the patient catheter. It is also to be understood that the specific devices and processes illustrated in the attached drawings, and described in the following specification, are simply exemplary embodiments of the disclosure. Hence, specific dimensions and other physical characteristics related to the embodiments disclosed herein are not to be considered as limiting.

The present disclosure is generally directed to embodiments of single-use medical connectors including, for example, a male luer connector configured to be inserted into and engaged to a female luer connector to form a fluid-tight connection therebetween. The connectors can include locking and/or shielding mechanisms for preventing inadvertent reuse of the single-use connector. In some instances, the locking or shielding structures can also be configured to reinforce the luer connection between the male and female luer connectors by, for example, providing a compression fitting pressing the luer connectors towards one another. For example, as described herein, a guard or shield extending from the female luer connector may be configured to engage a portion of the male luer connector and to apply a biasing force to the male luer connector, thereby drawing the male luer connector into the female luer connector. The present disclosure is also directed to patient sets, tubing sets, and fluid injection systems including one or more single-use connectors with structures for preventing the single-use connector from being reconnected to another connector.

### Connector Assemblies

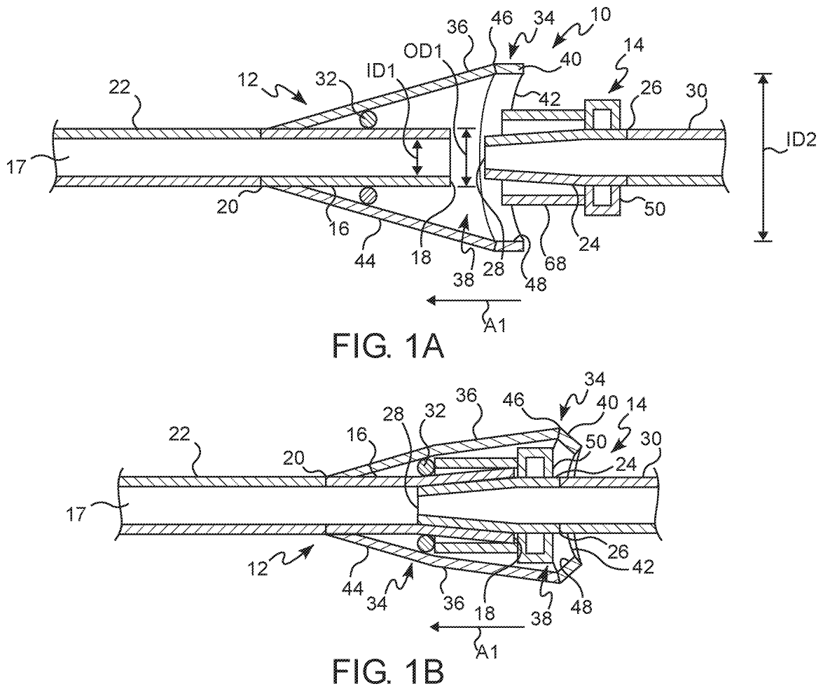

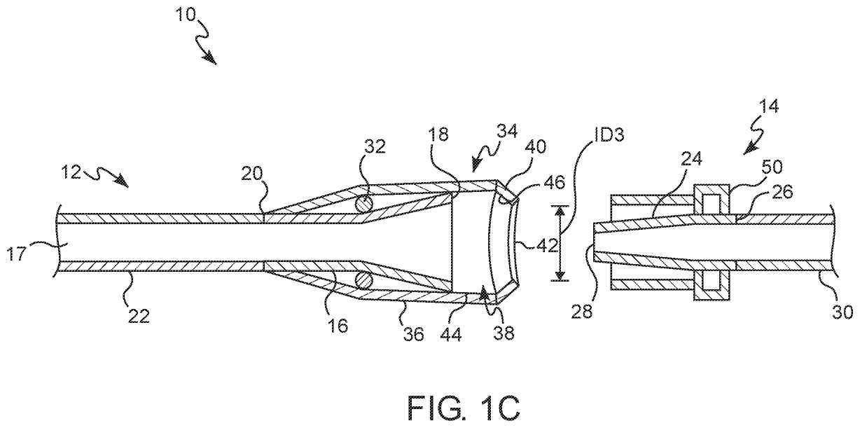

Embodiments of medical connector assemblies 10 including a single-use connector 12 removably connected to a multi-use or reusable connector 14 are shown in FIGS. 1A-4C, in which like reference numbers refer to like features. The single-use connector 12 includes a hollow, tubular housing 16, defining a lumen 17 and having an open proximal side 18 opposite a distal side 20 along a longitudinal length of the housing 16. A fluid conveying conduit, such as flexible medical tubing 22, extends from the distal side 20 of the housing 16. The tubing 22 can be connected, for example, to a catheter or vascular access device for enabling a medical fluid to flow from a fluid delivery device or injector to the patient through the connector assembly 10 and tubing 22. In a similar manner, the multi-use or reusable connector 14 can include a hollow, tubular housing 24, separate from the housing 16 of the single-use connector 12, having a proximal side 26 opposite a distal side 28 along a longitudinal length of the housing 24. Flexible tubing 30 extends from the proximal side 26 of the reusable connector 14. The flexible tubing 30 can extend, for example, from the reusable connector 14 to a valve manifold, a fluid reservoir, and/or a syringe of a fluid injector or drug delivery device. As shown in FIG. 1B, the open proximal side 18 of the single-use connector 12 is connectable to the distal side 28 of the reusable connector.

The housings 16, 24 are generally formed primarily from a rigid material, such as injection molded plastic. In other examples, portions of the housings 16, 24 could be formed from medical grade glass or metal. The housings 16, 24 can be formed from a substantially transparent or translucent material so that a user can visually confirm that the connectors 12, 14 are firmly connected together. The transparent or translucent housings 16, 24 also allow users to visually confirm when fluid passes through the tubing 22, 30 and connectors 12, 14.

The housings 16, 24 can be sized to be connected together and to form a substantially fluid-tight seal to permit leak-free fluid flow through the connector assembly 10. In general, the size of the housings 16, 24 is selected based on sizes of other components of a fluid injection system or drug delivery device. For example, dimensions of the housing(s) 16, 24 can be selected based on the size of the medical tubing or of fluid inlet and/or outlet ports of a medical injector to which the connector assembly 10 is attached. When used with conventional medical tubing having an outer diameter of 0.75 inch, the housings 16, 24 can have a length from 1.0 inch to 4.0 inches. The open proximal side 18 of the single use connector 12 can, for example, have an inner diameter ID1 of about 0.80 inch and an outer diameter OD1 of about 1.0 inch. The distal side 28 of the reusable connector 14 can have an outer diameter slightly smaller than the inner diameter ID1 of the open proximal side 18 so that it can be inserted in the open proximal side 18 of the single-use connector 12.

In some examples, the connectors 12, 14 are luer connectors. For example, the open proximal side 18 of the single use connecter 12 can be a thread-less attachment member (e.g., a female luer connector) configured to be connected to a thread-less attachment member (e.g., a male luer connector) of the distal side 28 of the reusable connector 14 to establish a fluid connection between the single-use connector 12 and the reusable connector 14. In other examples, the attachment members can be a snap fit member or locking structure, as are known in the art, for removably connecting the connectors 12, 14 together.

In order to further enhance the connection between the connectors 12, 14, the reusable connector 14 can include a shroud 68 configured to receive the open proximal side 18 of the single-use connector 12. In some examples, friction between the inner surface of the shroud 68 and outer surface of the proximal side 18 can be sufficient to produce a suitable connection. In other examples, an inner surface of the outer shroud 68 can include helical threads configured to engage corresponding helical threads on an outer surface of the proximal side 18 of the single-use connector 12. However, it is believed that thread-less attachment members are preferable for many applications, since the connectors can be joined together and released more quickly. In contrast, with threaded attachment members, the user is required to twist one of the connectors 12, 14 relative to the other connector 12, 14 to establish the threaded connection between the attachment members. In some examples, a seal, such as an O-ring 32, surrounds or partially surrounds the housing 16 of the single-use connector 12 to further enhance the connection and seal between the connectors 12, 14.

In some examples, the assembly 10 also includes at least one guard member 34 connected to the tubular housing 16 of the single-use connector 12. The at least one guard member 34 can be configured to transition between a pre-use or open position (shown in FIG. 1A) in which the single-use connector 12 can be connected to the reusable connector 14, an in-use or connected position (shown in FIG. 1B), and a post-use or closed position (shown in FIG. 1C) in which the guard member 34 prevents the single-use connector 12 from being re-connected to the reusable connector 14. In order to prevent reuse of the single-use connector 12, removing or disconnecting the single-use connector 12 from the reusable connector 14 causes the at least one guard member 34 to transition to the closed position.

The at least one guard member 34 can be any suitable structure for blocking the connectors 12, 14 from being attached to one another to prevent reuse of the single-use connector 12. In some embodiments, as shown, for example, in FIGS. 1A-3B, the at least one guard member 34 is integral with or mounted to the housing 16 of the single-use connector 12. In other examples, the at least one guard member 34 is integrally formed with or mounted to the housing 24 of the reusable connector 14. In addition to blocking the single-use connector 12 from being connected to the reusable connector 14, the at least one guard member 34 can also be configured to provide a visual indication that the single-use connector 12 has already been used and should not be reconnected to the reusable connector 14. For example, the at least one guard member 34 may include portions which, at least in the closed position (shown, for example, in FIG. 1C), conspicuously cover the proximal open side 18 of the single-use use connector 12 and/or the distal side 20 of the reusable connector 14 to clearly inform the user that a single-use connector 12 has already been used and should not be connected to the reusable connector 14. In some examples, the single-use connector 12 can include additional visual indicators confirming that the single-use connector 12 has already been used. For example, portions of the housing 16 or medical tubing 22 can include color-change regions, which change color when attached to the reusable connector 14 or when fluid passes through the connector assembly 10. In order to produce such color changes, for example, portions of the housing 16 could be impregnated with chemicals which change color in the presence of fluid. In other examples, portions of the housing 16 could be impregnated with a chemical composition that changes color when brought in proximity to the reusable connector 14. If the color-change portion of the single-use connector 12 has changed color, the user may be instructed that the connector 12 has already been used and should be discarded. In a similar manner, the open proximal side 18 of the housing 16 could include a pigment or painted portion, which changes appearance when contacted by the outer surface of the reusable connector 14. For example, the outer surface of the reusable connector 14 may scratch or otherwise change the appearance of the pigment or painted portion, indicating that the single use connector 12 has come into contact with a reusable connector 14.

With specific reference to FIGS. 1A-1C, in some examples, the at least one guard member 34 includes an annular compression sleeve 36 at least partially enclosing the open proximal side 18 of the housing 16. As shown in FIG. 1B, the compression sleeve 36 engages the reusable connector 14 to secure and/or reinforce the connection between the single use connector 12 and the reusable connector 14. Accordingly, the compression sleeve 36 functions as a compression fitting which draws the reusable connector 14 into the single use connector 12. The compression sleeve 36 can be formed from a variety of materials depending on the structure and intended use of the sleeve 36. For example, the compression sleeve 36 can be formed from a rigid plastic material having one or more flex points or joints positioned and biased so that the compression sleeve 36 naturally adopts the closed position (shown in FIG. 1C), when the single-use connector 12 is removed from the reusable connector 14. In other examples, the compression sleeve 36 can be formed from an elastomeric material configured to wrap around and at least partially cover the open proximal side 18 of the housing 16 when in the closed position.

In some examples, the compression sleeve 36 defines an interior 38 and a first or proximal portion 40 defining a proximal opening 42 having a diameter ID2, positioned such that the distal side 28 of the reusable connector 14 can be inserted into the interior 38 of the compression sleeve 36 through the proximal opening 42 and connected to the proximal side 18 of the single-use connector 12 when the at least one guard member 34 is in the pre-use or open position (shown in FIG. 1A). In the post-use or closed position (shown in FIG. 1C), the inner diameter ID2 of the opening 42 contracts, thereby preventing the single-use connector 12 from being reconnected to the reusable connector 14. The compression sleeve 36 can also include a second or distal annular portion 44 mounted to and extending proximally and radially outwardly from the housing 16 of the single-use connector 12. The proximal portion 40 of the compression sleeve 36 is connected to and extends proximally and radially inwardly from the distal portion 44 of the compression sleeve 36. The annular distal portion 44 and annular proximal portion 40 can be connected at an annular joint 46 which permits the proximal portion 40 to pivot relative to the distal portion 44. The annular joint 46 can be a scored region or groove that permits the proximal portion 40 of the sleeve 36 to flex, bend, or pivot radially inwardly (in the closed position) and outwardly (in the open position) as shown in FIGS. 1A-1C. For example, in the pre-use or open position (shown in FIG. 1A), the proximal portion 40 of the compression sleeve 36 can be angled so that a diameter of the opening 42 is large enough to receive the reusable connector 14. In the post-use or closed position (shown in FIG. 1C), the annular proximal portion 40 can be angled radially inwardly to reduce a diameter of the opening 42 and to prevent a user from inserting the used single-use connector 12 into the reusable connector 14.

When the connectors 12, 14 are attached together, as shown in FIG. 1B, the compression sleeve 36 engages at least a portion of the reusable connector 14 to reinforce the connection between the single-use connector 12 and the reusable connector 14. For example, an inner surface 48 of the proximal portion 40 of the compression sleeve 36 can be configured to contact a proximal facing portion 50 or surface of the housing 24 of the reusable connector 14 to bias the reusable connector 14 into the single-use connector 12 in a direction of arrow A1. In the post-use or closed position (shown in FIG. 1C), the annular distal portion 44 and/or the annular proximal portion 40 of the compression sleeve 36 are biased radially inwardly reducing the diameter ID3 (shown in FIG. 1C) of the opening 42 of the compression sleeve 36, thereby blocking the user from inserting the single-use connector 12 back into the reusable connector 14.

In use, in order to connect the single-use connector 12 to the reusable connector 14, the user first ensures that the single-use connector 12 is in the pre-use or open position meaning that it has not been previously connected to a reusable connector 14. For example, the user can look for conspicuous visual indicators (e.g., color change portions or painted portions) of the single-use connector 12 and/or at least one guard member 34 to confirm that the single-use connector 12 has not been previously used.

In order to attach the single-use connector 12 to the reusable connector 14, the user aligns the open proximal side 18 of the single-use connector 12 with the distal side 28 of the reusable connector 14. For example, the user may line up different locking structures or guide ribs of the single-use connector 12 with corresponding locking structures or grooves of the reusable connector 14. The user would then push the single-use connector 12 in an axial direction, opposite relative to that shown by arrow A1, to connect the single-use connector 12 to the reusable connector 14.

To disconnect the single-use connector 12 from the reusable connector 14, the user pulls the single-use connector 12 axially away from the reusable connector 14. For example, the user may grasp the housing 16 of the single-use connector 12 in one hand and the housing 24 of the reusable connector 14 in the other hand, and then move his/her hands and the housings 16 and 24 therewith away from one another. As described herein, disconnecting the single-use connector 12 from the reusable connector 14 causes the at least one guard member 34 of the single-use connector 12, such as the compression sleeve 36, to transition to the post-use or closed position to prevent reuse of the single-use connector 12.

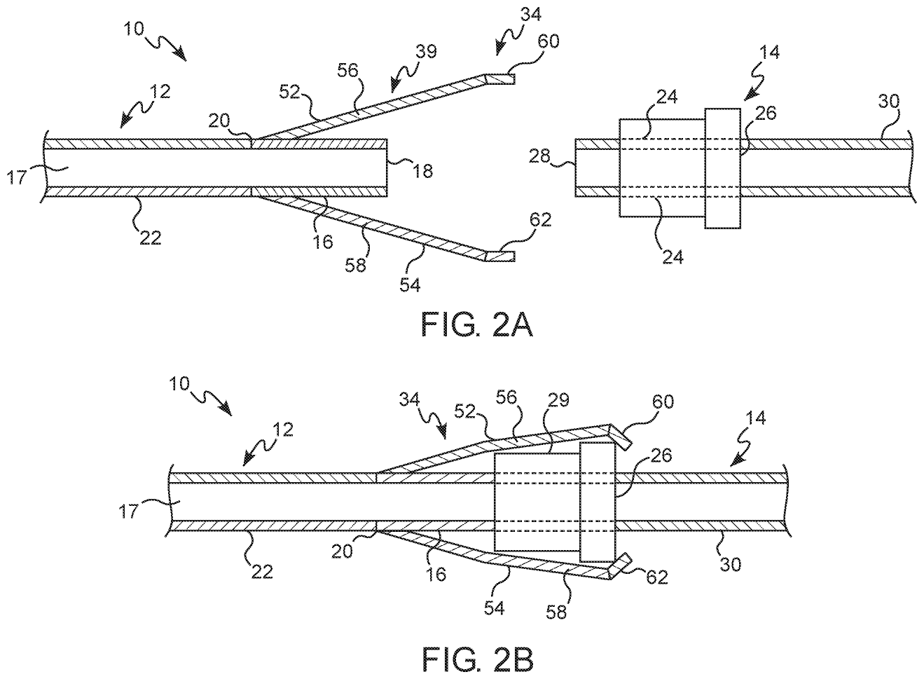

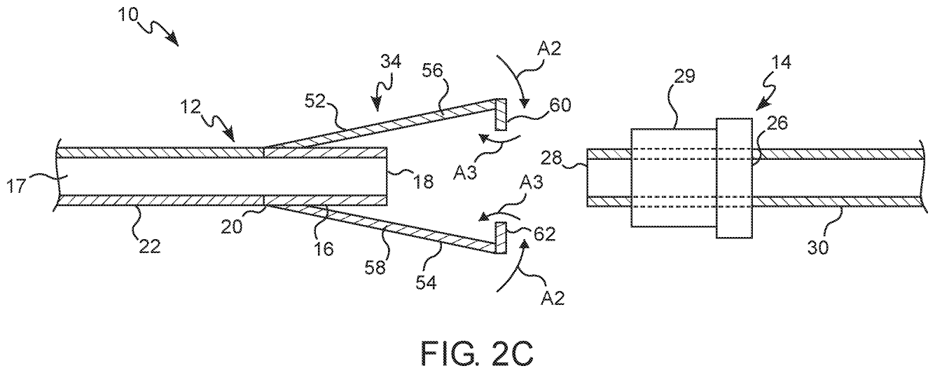

Another embodiment of a medical connector assembly 10, shown in FIGS. 2A-2C, includes the single-use connector 12, at least one guard member 34, and the reusable connector 14. The single use connector 12 and the reusable connector 14 each include hollow, tubular housings 16, 24 which are similar in size, shape, and material composition to housings of previously described examples. Also as in previously described examples, the connector(s) 12, 14 are configured to transition from a pre-use or open position (shown in FIG. 2A) in which the single-use connector 12 is ready to be connected to the reusable connector 14, to an in-use or connected position (shown in FIG. 2B) in which the single-use connector 12 is connected to the reusable connector 14, and ultimately to a post-use or closed position (shown in FIG. 2C) in which the single use connector 12 has been disconnected from reusable connector 14 and is thereafter blocked by the at least one guard member 34 from reconnecting to the reusable connector 14.

As shown in FIGS. 2A-2C, the at least one guard member 34 includes at least one first arm 52 and at least one second arm 54 extending from the housing 16 of the single-use connector 12. The arms 52, 54 are elongated members extending from the housing 16 and can include joints or pivot points for positioning the arms 52, 54 to engage the reusable connector 14 and/or to block the single use connector 12 from being reconnected to the reusable connector 14. In some examples, the arms 52, 54 are integrally molded with other portions of the housing 16. In other examples, the arms 52, 54 can be attached to the housing 16 by a fastener or suitable adhesive. The arm(s) 52, 54 can be formed from a rigid material, such as plastic or metal. Desirably, the arm(s) 52, 54 are sufficiently rigid so that, when in the post-use or closed position (shown in FIG. 2C), the arms 52, 54 block a previously used single-use connector 12 from being mounted to a reusable connector 14.

In some examples, the first arm 52 and the second arm 54 each include a first portion or segment 56, 58 connected to and extending proximally and radially outwardly from the housing 16 of the single-use connector 12 and a second portion or segment 60, 62 connected to and extending proximally and radially inwardly from the first segment 56, 58 of each arm 52, 54. In some examples, the first segment 56, 58 is biased towards the post-use or closed position, shown in FIG. 2C. As shown in FIG. 2B, when the single user connector 12 is connected to the reusable connector 14, an inwardly directed surface of the second segment 60, 62 of each arm 52, 54 engages a portion of the reusable connector 14 and exerts a compressive force on the reusable connector 14 to press the reusable connector 14 into the single-use connector 12, thereby reinforcing the connection between the single-use connector 12 and the reusable connector 14.

Disconnecting the single-use connector 12 from the reusable connector 14 causes the arms 52, 54 to transition from the in-use or connected position (shown in FIG. 2B) to the post-use or closed position (shown in FIG. 2C). More specifically, disconnecting the single-use connector 12 from the reusable connector 14 causes the arms 52, 54 to pivot radially inwardly relative to the housing 16 of the single-use connector 12. The second portion or segment 60, 62 of the arms 52, 54 may also rotate or pivot in a direction of arrow A3 (shown in FIG. 2C). As a result of such movement of the arms 52, 54, an outer surface of the second portion or segment 60, 62 of the arms 52, 54 is moved to block reconnection of the single-use connector 12 with the reusable connector 14. The repositioned and extended arm(s) 52, 54 also provide a clear visual indication for a user that the single-use connector 12 has already been used, helping the user to recognize that the used single use connector 12 should not be connected to a reusable connector 14.

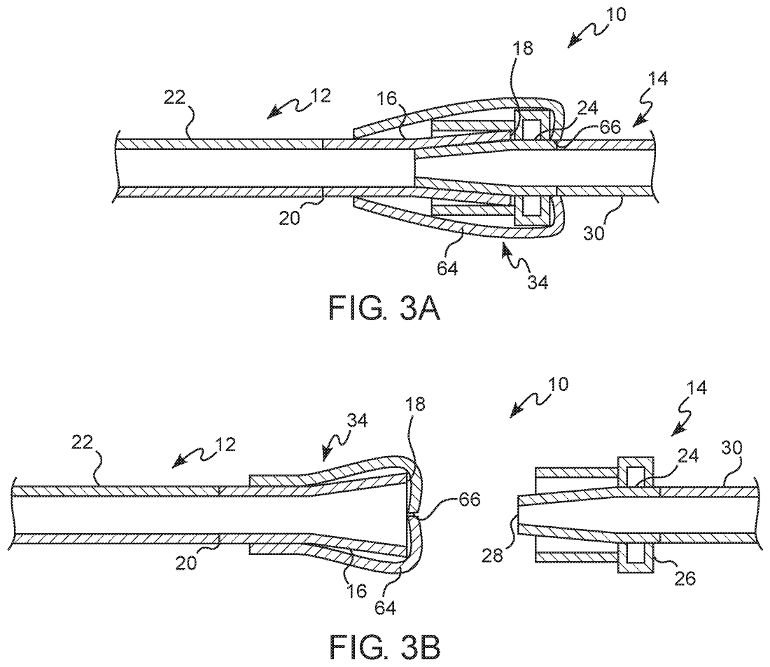

FIGS. 3A and 3B show another embodiment of a medical connector assembly 10 including the single use connector 12 that is connectable to a reusable connector 14. As in previous examples, the connectors 12, 14 include hollow, tubular housings 16, 24 connected, at one end, to segments of flexible tubing 22, 30, respectively For example, the reusable connector 14 can include a proximal end or side 26 connected to the flexible tubing 30 and a distal end or side 28 that is designed to be connected to the single-use connector 12. The single-use connector 12 includes a distal side 20 connected to the flexible tubing 22 and an open proximal side 18 that is designed to be connected to the reusable connector 14. The hollow, tubular housings 16, 24 of the connectors 12, 14 are generally similar in size, shape, and material composition to previously described connectors.

The medical connector assembly 10 also includes at least one guard member 34. In this embodiment, the at least one guard member is an elastomeric compressive fitting or sleeve 64 connected to the tubular housing 16 of the single-use connector 12. The elastomeric sleeve 64 is configured to transition between a pre-use or open position (not shown) in which the single-use connector 12 is ready to be connected to the reusable connector 14, to an in-use or connected position (shown in FIG. 3A), and ultimately to a post-use or closed position (shown in FIG. 3B) in which the single use connector 12 has been disconnected from reusable connector 14 and the elastomeric sleeve 64 prevents the single-use connector 12 from being re-connected to the reusable connector 14.

Similar to previous examples, the elastomeric sleeve 64 is connected to the housing 16 of the single-use connector 12 and extends proximal and radially outwardly therefrom. In the pre-use or open position, the elastomeric sleeve 64 defines a proximal opening 66 of sufficient size to receive the housing 24 of the reusable connector 14. Inserting the reusable connector 14 into the elastomeric sleeve 64 of single-use connector 12 releases the sleeve 64 from the pre-use or open position, thereby allowing the sleeve to compress against or cinch about the housings 16, 24 of the connectors 12, 14 to secure or reinforce the attachment between the connectors 12, 14, as shown in FIG. 3A.

To disconnect the single-use connector 12 from the reusable connector 14, the user pulls the connectors 12, 14 axially away from each other. Pulling the connectors 12, 14 away from each other stretches the elastomeric sleeve 64 axially and radially outwardly, thereby increasing the diameter of the proximal opening 66 of the sleeve 64 so that the reusable connector 14 can be removed from the elastomeric sleeve 64. Once the reusable connector 14 is removed from the elastomeric sleeve 64, the sleeve 64 contracts around the open proximal side 18 of the housing 16 of the single use connector 12 as shown in FIG. 3B. In this position, the elastomeric sleeve 64 covers at least a portion of the open proximal side 18 of the housing 16, thereby preventing the user from reconnecting the used single-use connector 12 to a reusable connector 14.

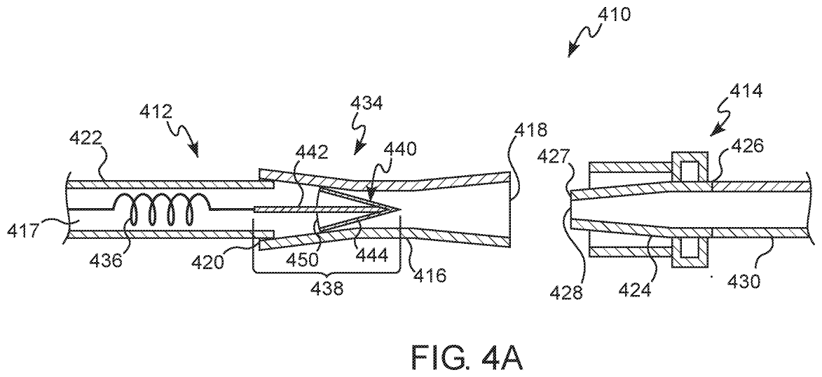

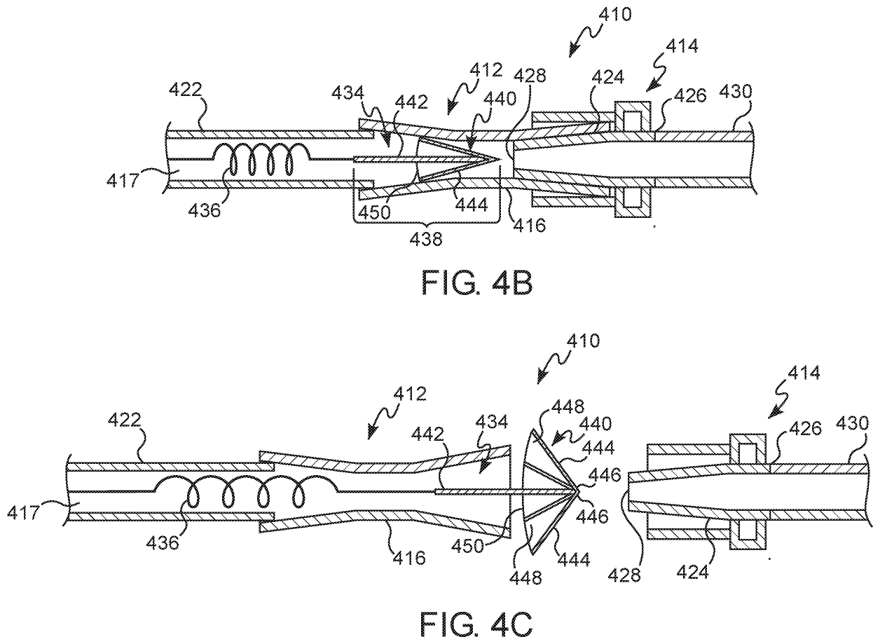

FIGS. 4A-4C illustrate another exemplary embodiment of a medical connector assembly 410 including a single use connector 412 that is connectable to a reusable connector 414. As in previous examples, the connectors 412, 414 include hollow, tubular housings 416, 424 connected, at one end, to flexible tubing 422, 430. For example, the reusable connector 414 can include a proximal end or side 426 connected to the flexible tubing 430 and a distal end or side 428 that is designed to be connected to the single-use connector 412. The single-use use connector 412 includes a distal side 420 connected to the flexible tubing 422 and an open proximal side 418 that is designed to be connected to the reusable connector 414. The hollow, tubular housings 416, 424 of the connectors 412, 414 are generally similar in size, shape, and material composition to previously described connectors.

The medical connector assembly 410 also includes at least one guard member 434 connected to the tubular housing 416 of the single-use connector 412. In this embodiment, the at least one guard member 434 is configured to transition between a pre-use or open position (shown in FIG. 4A) in which the single-use connector 412 is ready to be connected to the reusable connector 414, to an in-use or connected position (shown in FIG. 4B) in which the connectors 412, 414 are attached together, and ultimately to a post-use or closed position (shown in FIG. 4C) in which the single use connector 412 has been disconnected from reusable connector 414 and the at least one guard member 434 prevents the single-use connector 412 from being re-connected to the reusable connector 414. In the embodiment of FIGS. 4A-4C, the at least one guard member 434 is positioned in a lumen 417 defined by an interior sidewall of the housing 416 of the single-use connector 412. In the pre-use or open position (shown in FIG. 4A) and the in-use or connected position (shown in FIG. 4B), the guard member 434 is fully retracted in the housing 416, so that the housing 416 of single-use connector 412 can be connected to the reusable connector 414. In the post-use or closed position as described below in connection with FIG. 4C, the proximal tip of guard member 434 extends from the open proximal side 418 of the single-use connector 412 to prevent the user from reconnecting the single-use connector 412 to a reusable connector 414.

In one non-limiting aspect of medical connector 410, the guard member 434 can include a spring 436, a deployable support or basket 440 and an elongated member or rod 442 connected therebetween, the latter two of which forming an extendable portion 438 as best shown in FIGS. 4A-4B. In operation, the spring 436, when released, advances the extendable portion 438 of the guard member 434 proximally to the closed position. As a result, as the deployable basket 440 at the proximal tip of guard member 434 emerges from the proximal end of housing 416, the deployable support or basket 440 transitions from a retracted position to a deployed position. In some examples, the deployable support or basket 440 is an umbrella structure including a plurality of flexible frame members 444. The frame members 444 have a first end 446 pivotally connected to the rod 442 and a free second end 448 opposite the first end 446 (shown in FIG. 4C). Similar to an umbrella, the deployable support or basket 440 can also include, as best shown in FIG. 4A, a cover 450 formed, for example, from a fabric or a plastic film, attached to and extending across the plurality of frame members 444.

Although generally biased to the deployed position by virtue of spring 436 and elongated member or rod 442, the deployable support or basket 440 is normally maintained in its retracted position by a narrowed inner surface of the sidewall of the housing 416, as shown in FIGS. 4A and 4B. Relatedly or alternatively, in the in-use or connected position (shown in FIG. 4B), a stop member 427 on the distal side 428 of the reusable connector 414 can contact the basket 440 to counteract the biasing force of the spring 436 and thereby maintain the guard member 434 in the housing 416. When the single use connector 412 is removed from the reusable connector 414, the spring 436 releases, thus pushing the elongated member or rod 442 proximally and causing the deployable support or basket 440 to advance through and extend from the proximal end of housing 416. Once free from the housing 416, the deployable support or basket 440 is able to deploy, such that a maximum outer diameter of the basket 440 increases to block the single-use connector 412 from being reconnected to a reusable connector 414. When the rod 442 is extended and the basket 440 is in the deployed position, the single use connector 412 cannot be connected to a reusable connector 414.

### Fluid Injection Systems

The single-use connectors disclosed herein can be adapted to the fluid inlet or outlet ports typically found in many automated fluid injectors and their associated tubing sets. As is well known in the art, many fluid injection systems employ a reusable or multi-use disposable set (MUDS) to administer one or more fluids to multiple patients over the course of multiple injection procedures along with a single-use disposable tubing set (SUDS) for each patient and in each such procedure. One such multiple fluid injection system including a MUDS for use therewith is described in United States Patent Appl. Pub. No. 2018/0015274A1 to Haury et al., titled “Multiple Fluid Delivery System With Multi-Use Disposable Set And Features Thereof,” and such a single-use disposable set (SUDS) including a patient fluid line capable of being adapted to include the single-use connectors disclosed herein is described in United States Patent Appl. Pub. No. 2016/0331951A1 to Sokolov et al., titled “Single-use Disposable Set Connector,” both references assigned to the assignee of the present invention and hereby incorporated by reference herein. The use of such a SUDS for each patient assures sterility of the MUDS and avoids contamination of the fluid(s) as the injection procedures are performed in sequence with the MUDS. As will be described below, the single-use connectors described herein can be adapted to be connected to and/or integrally formed with such SUDS devices.

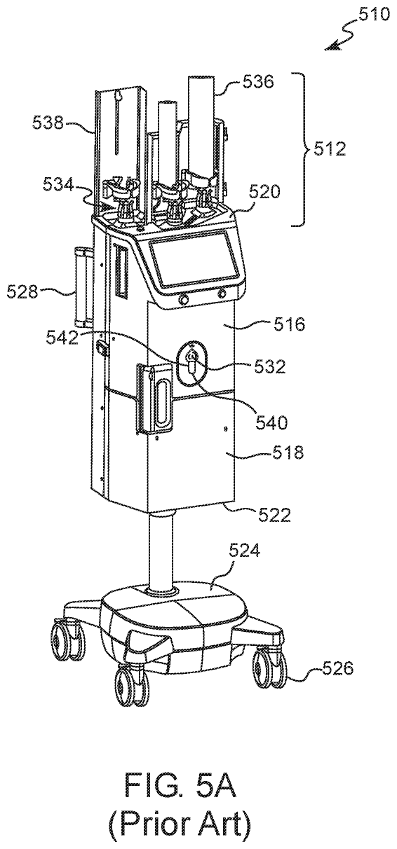

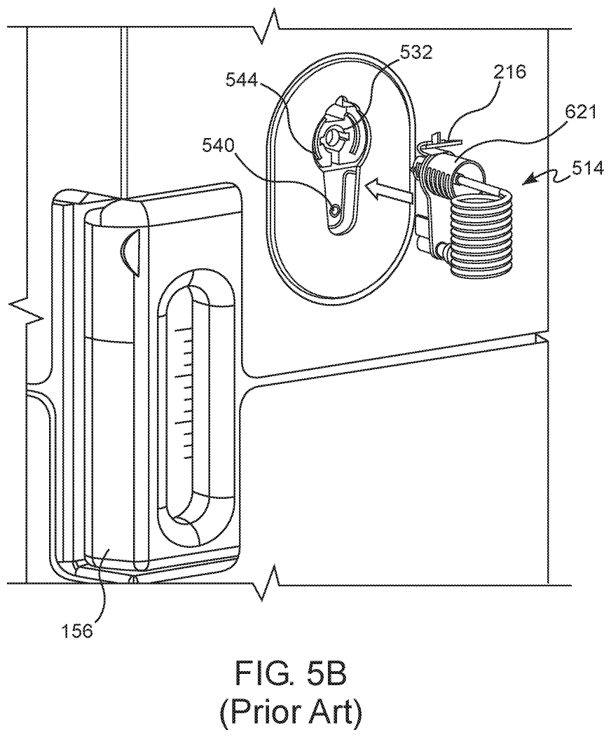

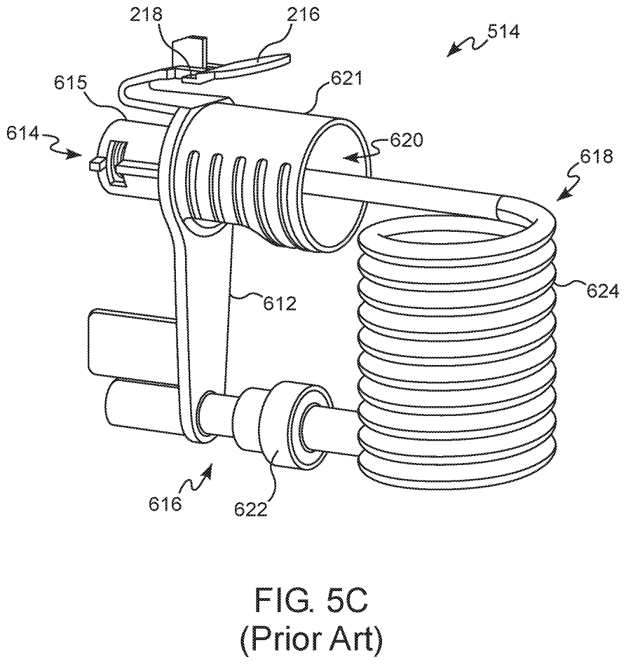

As shown in FIGS. 5A-5C, a multi-fluid medical injector/injection system 510 (hereinafter “fluid injector system 510”) includes a powered injector apparatus or device and a fluid delivery set(s) intended to be associated with the injector to deliver one or more fluids from one or more multi-dose containers under pressure into a patient. For example, the system 510 can include a multi-patient disposable set (MUDS) 512 removably enclosed within an enclosure 516 of the fluid injector system 510. A single-use disposable set (SUDS) 514 can be removably connected to the MUDS 512 through a port, opening, or slot 544 in the enclosure 516. The SUDS 514 (shown in FIGS. 5B and 5C) is configured to deliver fluid from the MUDS 512 to a vascular access site and/or catheter of a patient through one or more connectors and segments of flexible medical tubing as described herein.

In some examples, the injector enclosure 516 includes opposed lateral sides 518, an upper end 520, and a lower end 522. In some embodiments, the enclosure 516 may be supported on a base 524 having one or more wheels 526 for rotatable and movable support of the enclosure 516 on a floor of an imaging suite. The one or more wheels 526 may be lockable to prevent the enclosure 516 from inadvertently moving once positioned at a desired location. At least one handle 528 may be provided to facilitate moving and positioning the fluid injector system 510. In other embodiments, the enclosure 516 may be removably or non-removably secured to a fixed surface, such as a floor, ceiling, wall, or other structure. The enclosure 516 encloses the various mechanical, electrical and power components necessary to drive the mechanical drive components. It also encloses control components, such as electronic memory and electronic control devices (hereinafter electronic control device(s)), used to control operation of the mechanical drive components (e.g., movable piston elements) associated with the fluid injector system 510. Such piston elements may be reciprocally operable via electro-mechanical drive components such as a ball screw shaft driven by a motor, a voice coil actuator, a rack-and-pinion gear drive, a linear motor, and the like. In some embodiments, at least some of the mechanical, electrical and power components, and the control components, may be provided in or on the base 524.

The fluid injector system 510 further includes at least one bulk fluid connector 534 for connection with at least one bulk fluid source 536. In some embodiments, a plurality of bulk fluid connectors 534 may be provided. For example, as shown in FIG. 5A, three bulk fluid connectors 534 may be provided in a side-by-side or other arrangement. In some embodiments, the at least one bulk fluid connector 534 may be a spike configured for removably connecting to the at least one bulk fluid source 536, such as a vial, a bottle, or a bag. The at least one bulk fluid connector 534 may have a reusable or non-reusable interface with each new bulk fluid source 536. The at least one bulk fluid source 536 may be configured for receiving a medical fluid, such as saline, contrast solution, or other medical fluid, for delivery to and use by the fluid injector system 510. The enclosure 516 may have at least one support member 538 for supporting the at least one bulk fluid source 536 once it is connected to the fluid injector system 510.

The at least one bulk fluid connector 534 may be formed on the MUDS 512 or may be separate from and fluidly connected to the MUDS 512. In either case, the MUDS 512 is configured to deliver fluid from the fluid source(s) to the SUDS 514 through a suitable fluid connection port 532. The MUDS 512 also includes a waste reservoir and waste inlet port 540 for receiving waste fluid from the SUDS 514 during a priming procedure.

In some examples, the connection port 532 and waste inlet port 540 are shielded by at least a portion of the enclosure 516 of the fluid injector system 510. For example, the ports 532, 540 may be recessed within a slot 544 in the enclosure 516 configured to receive the SUDS 514, as shown in FIG. 5B. Recessing the ports 532, 540 within the enclosure 516 may serve to preserve the sterility of the ports 532, 540 by preventing or limiting a user or patient from touching and potentially contaminating the ports 532, 540 or portions thereof, which may come into contact with the fluid to be injected to the patient. In other examples, the connection port 532 may be formed directly on the enclosure 516 and connected to the MUDS 512 by a fluid path (not shown). Desirably, the connection between the SUDS 514 and the ports 532, 540 is a releasable connection to allow the SUDS 514 to be selectively connected and disconnected from the ports 532, 540.

### Single-Use Disposable Connector Sets (SUDS)

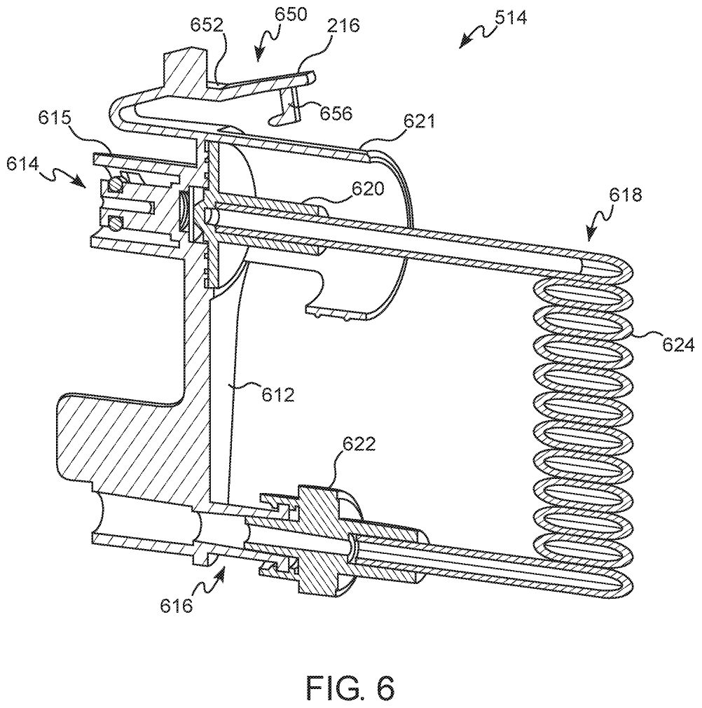

With reference to FIGS. 6-9C, various new embodiments of a SUDS 514 of the type configured to be mounted to a MUDS 512 of the injector system 510 are illustrated. It should be understood that, like the prior art version of SUDS 514 shown in FIGS. 5B and 5C, the SUDS 514 of each new embodiment herein described is, prior to use, desirably packaged in a pre-sterilized, sealed package that protects the SUDS 514 from contamination with air or surface-borne contaminants. Alternatively, the sealed package and SUDS 514 therein may be sterilized after packaging using, for example, electron beam (E-beam) sterilization techniques.

As shown in FIGS. 5B and 5C, the SUDS 514 includes a housing 612 configured to be inserted into a corresponding slot or port, such as the slot 544, of the injector system 510 and into the MUDS 512, as best shown in FIGS. 5A and 5B. Specifically, a fluid inlet port 614 of the SUDS 514 extends through the housing 612 and is configured to removably engage at its proximal end with the connection port 532 of the MUDS 512. The SUDS 514 also includes a waste outlet port 616 extending through the housing 612. The waste outlet port 616 is configured to removably engage at its proximal end with the waste inlet port 540 of the MUDS 512 to establish a fluid connection therethrough with a waste reservoir 156, which may be either integral to or separate from the MUDS 512. The SUDS 514 also includes a patient fluid line 618 that, when positioned as shown in FIG. 5C, provides a fluid connection between the fluid inlet port 614 and the waste outlet port 616. In some examples, the patient fluid line 618 includes a fluid port connector 620 at its proximal end connected to the fluid inlet port 614, an end port connector 622 at its distal end connected to the waste outlet port 616, and flexible tubing 624 extending therebetween. The end port connector 622 is configured to be disconnected from the waste outlet port 616 and connected to a patient via, for example, a catheter, so that fluid can be provided from the fluid inlet port 614 to the patient. Meanwhile. the waste outlet port 616 remains engaged with the waste inlet port 540 of the MUDS 512. First and second skirts 615 and 621 extend around fluid inlet port 614 and fluid port connector 620, respectively, to prevent contact and contamination.

The SUDS 514 of the present invention can be implemented with one or more of the reconnection prevention mechanisms disclosed herein. According to one aspect of the present disclosure, the SUDS 514 can include various locking structures configured to prevent the SUDS 514 from being reused and/or reconnected to the MUDS 512 after an initial use.

For example, the fluid inlet port 614 can include one or more of the single use connectors shown in FIGS. 1A-4C. In that case, the guard members of the single-use connector described herein can be positioned to prevent the fluid inlet port 614 from being reconnected to the connection port 532 of the MUDS 512. For example, the fluid inlet port 614 could include the compressive sleeve 36 shown in FIGS. 1A-1C. In that case, disconnecting the fluid inlet port 614 from the connection port 532 of the MUDS 512 would cause the compressive sleeve 36 to transition to the post-use or closed position in which the fluid inlet port 614 is prevented from reconnecting to the connection port 532 of the MUDS 512. In some examples, the first skirt 615 surrounding the fluid inlet port 614 may include the compressive sleeve 36 for preventing the first skirt 615 from being reinserted or reconnected to the MUDS 512. In some examples, the waste outlet port 616 on the SUDS 512 may include the compressive sleeve 36 for preventing the waste outlet port 616 from being reinserted or reconnected to the waste inlet port 540 of the MUDS 512.

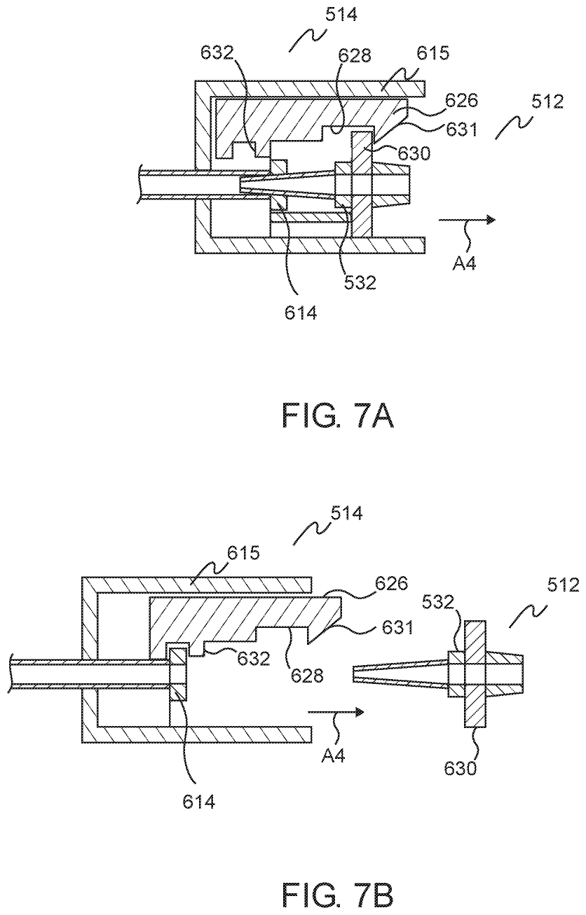

In some examples, the fluid inlet port 614 of the SUDS 514 may also include other guards, locking structures, or latches for preventing the fluid inlet port 614 from being reinserted or reconnected to the connection port 532 of the MUDS 512. An exemplary guard member 626 of the fluid inlet port 614 configured to prevent the fluid inlet port 614 from being reconnected to the connection port 532 of the MUDS 512 is illustrated in FIGS. 7A and 7B. The guard member 626 is an elongated member disposed in the fluid inlet port 614 and is configured to slide from a retracted position (shown in FIG. 7A) to an extended or blocking position (shown in FIG. 7B) in a longitudinal direction of the fluid inlet port 614.

As shown in FIG. 7A, when in the retracted position, the guard member 626 is configured to permit engagement of the fluid inlet port 614 of the SUDS 514 with the connection port 532 of the MUDS 512. For example, the guard member 626 may include a groove 628 configured to receive a corresponding annular lip or flange 630 of the connection port 532 of the MUDS 512. A ramp 631 on a distal surface of the guard member 626 may be configured to slide over the annular lip or flange 630, such as by being deflected due to contact with the annular lip or flange 630. After the annular lip or flange 630 passes over the ramp 631, the annular lip or flange 630 may be received within the groove 628 of the guard member 626, while the fluid inlet port 614 is connected with the connection port 532 of the MUDS 512, as shown in FIG. 7A. In order to disconnect the fluid inlet port 614 of the SUDS 514 from the connection port 532 of the MUDS 512, the user pulls the fluid inlet port 614 axially away from the connection port 532 in a direction of arrow A4. As a result of the engagement between the flange 630 and groove 628, pulling the end port connector 622 in the axial direction causes the guard member 626 to transition to an extended position, shown in FIG. 7B. In the extended position, a protrusion 632 of the guard member 626 extends over at least a portion of the fluid inlet port 614. In this position, the protrusion 632 blocks the fluid inlet port 614 to prevent the fluid inlet port 614 from being reconnected to the connection port 532 of the MUDS 512. In some examples, the first skirt 615 surrounding the fluid inlet port 614 may include the guard member 626 for preventing the first skirt 615 from being reinserted or reconnected to the MUDS 512. In some examples, the waste outlet port 616 on the SUDS 512 may include the guard member 626 for preventing the waste outlet port 616 from being reinserted or reconnected to the waste inlet port 540 of the MUDS 512.

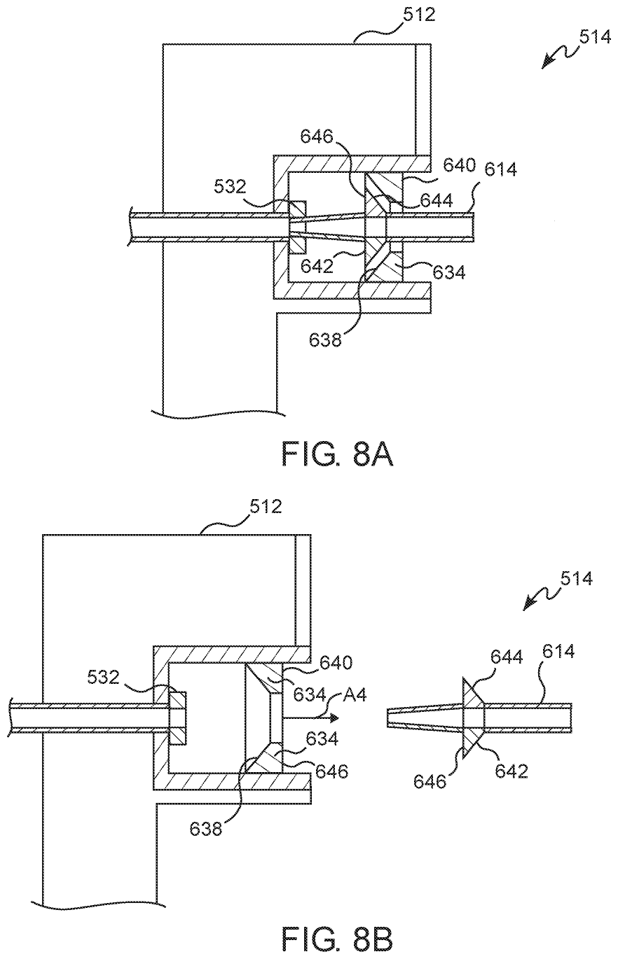

With reference to FIGS. 8A and 8B, another embodiment of a SUDS 514 is illustrated including structures for preventing the fluid inlet port 614 from being reconnected to the connection port 532 of the MUDS 512. As shown in FIGS. 8A and 8B, the connection port 532 of the MUDS 512 includes a ramped protrusion 634, extending radially inwardly from a sidewall of the connection port 532. The ramped protrusion 634 includes an inwardly directed surface 638 angled such that the first inlet port 614 can be removed from the connection port 532. The protrusion 634 also includes an outwardly directed surface 640 angled to block the first inlet port 614 from being reconnected to the connection port 532 after the fluid inlet port 532 has been removed from the connection port 532.

The first inlet port 614 can include a flange 642 having a distally facing angled surface 644 angled to correspond with the inwardly directed surface 638 of the ramped protrusion 634 of connection port 532. The flange 642 can also include a flat proximal surface 646 configured to engage the outwardly directed surface 640 of the ramped protrusion 634 of waste outlet port 616. The flange 642 may be movable between a first, retracted position, in which the distally facing angled surface 644 is positioned such that it is substantially parallel with an outer surface of the fluid inlet port 614 in order to allow insertion of the fluid inlet port 614 through the opening of the ramped protrusion 634, and a second, extended position, in which the distally facing angled surface 644 is positioned such that it is angled at an acute angle relative to the outer surface of the fluid inlet port 614. For example, the distally facing angled surface 644 can be angled by about 45 degrees, relative to a longitudinal axis of the fluid inlet port 614. In use, the distally facing angled surface 644 is initially in the first, retracted position to allow insertion of the fluid inlet port 614 through the opening of the ramped protrusion 634 and connection of the fluid inlet port 614 with the connection port 532. Once the fluid inlet port 614 is connected to the connection port 532, the distally facing angled surface 644 may be configured to transition to the second, extended position shown in FIG. 8A. In order to disconnect the fluid inlet port 614 from the connection port 532, a user pulls the fluid inlet port 614 axially away from the connection port 532. The distally facing angled surface 644 is configured to partially deform or bend when it contacts the inwardly facing angled surface 638 of the ramped protrusion 634 so that the fluid inlet port 614 can be easily removed from the connection port 532. Upon removal of the fluid inlet port 614 from the connection port 532, the distally facing angled surface 644 is configured to revert to the second, expanded position. In this position, the flat proximal facing surface 646 of the flange 642 is configured to prevent reinsertion of the fluid inlet port 614 into the connection port 532 due to contact the outwardly facing surface 640 of the ramped protrusion 634 with the flat proximal surface 646 of the flange 642.

In some examples, the first skirt 615 surrounding the fluid inlet port 614 may include the flange 642 for preventing the first skirt 615 from being reinserted or reconnected to the MUDS 512. In some examples, the waste outlet port 616 on the SUDS 512 may include the flange 642 for preventing the waste outlet port 616 from being reinserted or reconnected to the waste inlet port 540 of the MUDS 512.

As noted above, a SUDS that does not incorporate any of the reconnection prevention mechanisms taught herein is disclosed in U.S. Patent Application Publication 2016/0331951. That publication discloses, as is shown in FIGS. 5B and 5C herein, both a locking tab 216 whose proximal end is affixed to or formed as an integral part of skirt 621 of housing 612 and a receiving slot 544 corresponding thereto that is formed into the housing of MUDS 512. It also discloses a notch 218 or opening defined in each of the two sides of locking tab 216 and, relatedly, a prong corresponding to each notch 218 that protrudes from the receiving slot 544 of the MUDS. When the SUDS 514 is being installed into the MUDS 512, the locking tab 216 enters into the receiving slot 544 until its notches 218 are captured by the prongs that protrude from the receiving slot 544 at which point the SUDS 514 is connected to the MUDS 512. When SUDS 514 is placed in this secured or locked position, the fluid inlet port 614 and the waste outlet port 616 of the SUDS are connected, respectively, to the fluid connection port 532 and the waste inlet port 540 of the MUDS 512. Conversely, when the locking tab 216 is disengaged (e.g., depressed) so that its notches 218 are no longer retained by the corresponding prongs of the MUDS 512, the locking tab 216 can be removed from the receiving slot 544, and therewith the SUDS 514 from the MUDS 512, simply by pulling the former away from the latter. The locking tab 216 and its use in securing the SUDS 514 to, and releasing SUDS 514 from, the MUDS 512 is shown in the drawings of the aforementioned publication and also in FIGS. 5A-5C of the present application.

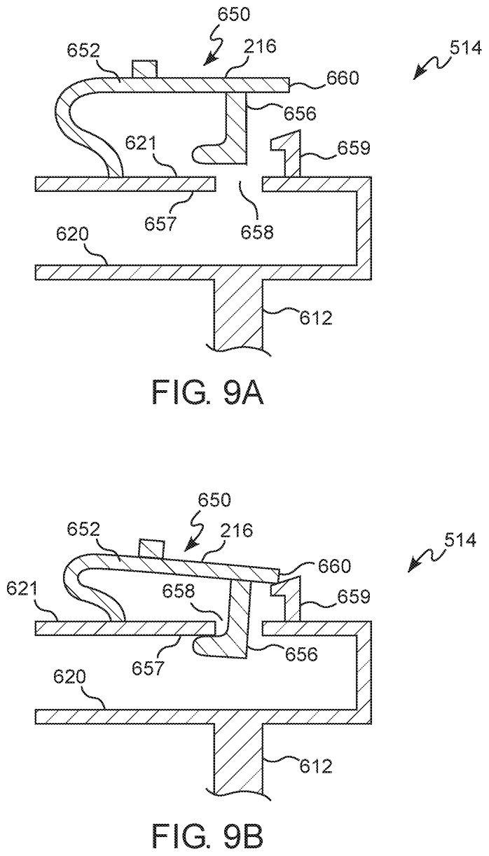

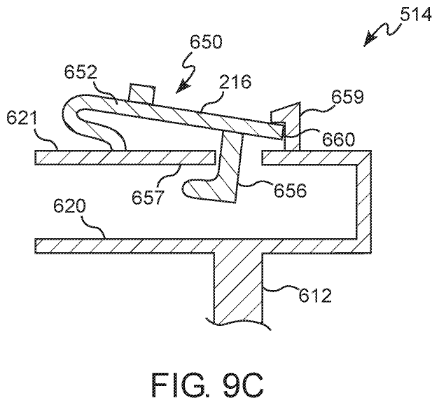

Referring again to the present disclosure, in some examples, the housing 612 of the SUDS 514 can include a latch mechanism for preventing the SUDS 514 from being reconnected to the MUDS 512. For example, as shown in FIGS. 6 and, 9A-9C, such a latch mechanism 650 may be implemented, at least in part, as a modification of the aforementioned locking tab 216 and the surrounding portions of housing 612. In particular, the latch mechanism 650 may include a flexible beam 652, a primary finger catch 656 affixed to the flexible beam, and a recess 658 defined within the skirt 621 to form a primary catch plate 657 therewith with which to catch the primary finger catch 656 as explained below. In this example, the latch mechanism 650 may also include a secondary finger catch 659 that is designed to be caught by a secondary catch plate 660; the secondary catch plate 660 preferably being manifested as the distal end of flexible beam 652/locking tab 216. This latch mechanism 650 and its finger catches 656/659 and catch plates 657/660 together form a reconnection prevention mechanism that is designed not only to work with locking tab 216 to enable SUDS 514 to be connected to and disconnected from MUDS 512 as previously described in connection with U.S. Patent Application Publication 2016/0331951 above but also to prevent SUDS 514 from thereafter being reconnected to MUDS 512 or any like-designed multi-use disposable set. As explained below, this reconnection prevention mechanism 650 is configured to transition from (i) a pre-use or open position (shown in FIG. 9A) in which SUDS 514 is allowed to be connected to MUDS 512 to (ii) an in-use or connected position (shown in FIG. 9B) in which SUDS 514 is connected to MUDS 512 and, ultimately, to (iii) a post-use or closed position (shown in FIG. 9C) in which SUDS 514 is prevented or blocked from being reconnected to MUDS 512 or any like-designed multi-use disposable set.

FIG. 9A shows SUDS 514 inclusive of the reconnection prevention mechanism incorporated therein in its pre-use or open position, i.e., prior to its insertion into a MUDS 512. In particular, the locking tab 216/flexible beam 652 is shown in an upward position to which its distal end is normally biased away from the skirt 621 of housing 612. As long as flexible beam 652 stays in this upward position, the primary finger catch 656 on the underside of beam 652 stays above recess 658 and disengaged from primary catch plate 657. As it is being installed into MUDS 512, however, the SUDS 514 begins its transition to the in-use or connected position, which is shown in FIG. 9B. In particular, this transition begins with locking tab 216 at its top surface initially contacting the prongs of MUDS 512 as the proximal end of locking tab 216 is being pushed into the receiving slot 544 of MUDS 512. As the locking tab 216 continues to be pushed into the receiving slot, the prongs of MUDS 512 ride along the top surface of tab 216 causing the flexible beam 652 thereof to be bent downwardly against the force of its normal upward bias and the primary finger catch 656 to be moved closer to engagement with primary catch plate 657. As the installation of SUDS 514 continues, the locking tab 216/flexible beam 652 soon bends to the point at which not only the primary finger catch 656 contacts and snaps past the top edge of primary catch plate 657 and into and through recess 658 but also the notches 218 in locking tab 216 reach the prongs of MUDS 512. At that point, due to the upward biasing force acting thereon, the locking tab 216/flexible beam 652 springs upwardly because its notches 218 have snapped up into and are captured by the prongs of MUDS 512. Also, having traveled past recess 658, the primary finger catch 656 catches upon the primary catch plate 657. This completes the connection of SUDS 514 to MUDS 512 and thus places SUDS 514 and its reconnection prevention mechanism 650 in the in-use or connected position shown in FIG. 9B.

FIG. 9C shows SUDS 514 and its reconnection prevention mechanism 650 in its post-use or closed position. In particular, to transition SUDS 514 from its in-use or connected position to its post-use or closed position, a user needs only to bend or push the locking tab 216/flexile beam 652 further downward to the point at which the secondary finger catch 659 catches, or becomes caught by, secondary catch plate 660. When the locking tab 216/flexible beam 652 is depressed or otherwise reaches that secondary catch point, two outcomes occur. First, the SUDS 514 becomes unlocked from MUDS 512 because the locking tab 216 has been moved downward to the point at which its notches 218 are freed from the corresponding prongs that protrude from the receiving slot 544 of the housing of MUDS 512. The allows the user to remove SUDS 514 from MUDS 512 simply by pulling the former away from the latter, as noted above. Second, the secondary finger catch 659 and secondary catch plate 660 are configured so that, once engaged as shown in FIG. 9C, they cannot be disengaged by the user. In particular, the locking tab 216 locks in place by virtue of its distal end (i.e., the secondary catch plate 660) being irreversibly trapped by or under the secondary finger catch 659. Despite the upward bias designed therein, the locking tab 216 becomes locked in a downward position wherein its notches 218 are rendered incapable of being again caught upon the prongs of MUDS 512. This prevents SUDS 514 from not only being reconnected to MUDS 512 but also from being connected to any like-designed multi-use disposable set.

While several embodiments of single-use connectors and/or multi-use connectors are shown in the accompanying figures and described hereinabove in detail, other embodiments will be apparent to, and readily made by, those skilled in the art without departing from the scope and spirit of the disclosure. For example, it is to be understood that this disclosure contemplates that, to the extent possible, one or more features of any embodiment can be combined with one or more features of any other embodiment.

Accordingly, the foregoing description is intended to be illustrative rather than restrictive.

What is claimed is:

1. A single-use medical connector comprising:
a housing configured to be inserted through a corresponding slot of a fluid injector for connection to a multi-use disposable set installed therein;
a fluid inlet port extending through the housing configured for removable engagement at a first end thereof with a fluid connection port of the multi-use disposable set to establish a fluid connection therewith;
a waste outlet port extending through the housing configured for removable engagement at a first end thereof with a waste inlet port of the multi-use disposable set to establish a fluid connection therewith; and
a patient fluid line providing a fluid connection between the fluid inlet port and the waste outlet port, the patient fluid line comprising a fluid port connector at a proximal end thereof connected to the fluid inlet port, an end port connector at a distal end thereof connectable to the waste outlet port, and flexible tubing extending between the fluid port connector and the end port connector,
wherein the end port connector is configured to be disconnected from the waste outlet port so that fluid can be provided from the fluid inlet port via the patient line to a patient while the waste outlet port remains engaged with the waste inlet port of the multi-use disposable set, and
a reconnection prevention mechanism configured to prevent reconnection of the fluid inlet port with the fluid connection port of the multi-use disposable set after the fluid inlet port is disconnected from the fluid connection port.

2. The single-use medical connector of claim 1, wherein the reconnection prevention mechanism comprises at least one guard member disposed on the fluid inlet port, the at least one guard member being configured to engage the connection port when the single-use medical connector is connected to the multi-use disposable set and, upon disconnecting the fluid inlet from the connection port, the at least one guard member is configured to transition to an extended position in which the at least one guard member blocks at least a portion of the fluid inlet port to prevent the fluid inlet port from being reconnected to the connection port.

3. The single-use medical connector of claim 1, wherein the housing of the single-use connector comprises a latch mechanism, the latch mechanism being configured to prevent the fluid inlet port from being reconnected to the fluid connection port of the multi-use disposable set after the fluid inlet port has once been engaged with and subsequently disengaged from the fluid connection port.

4. The single-use medical connector of claim 3, wherein the latch mechanism comprises a flexible beam which is depressed by a user to disconnect the housing from the multi-use disposable set and a finger catch that engages the housing when the flexible beam is depressed, thereby maintaining the latch mechanism in a blocking position in which the fluid inlet port is prevented from being reconnected to the fluid connection port.

5. The single-use medical connector of claim 1, wherein the fluid inlet port comprises a deflectable flange that is movable between a first, retracted position configured to permit connection of the fluid inlet port with the fluid connection port of the multi-use disposable set, and a second, extended position configured to prevent connection of the fluid inlet port with the fluid connection port of the multi-use disposable set.

6. The single-use medical connector of claim 5, wherein the deflectable flange comprises a distally facing angled surface angled such that the fluid inlet port can be removed from the fluid connection port and a flat proximal surface angled to block the fluid inlet port from reconnecting with the fluid connection port.

7. A single-use disposable set for use in administering fluid to a patient, the single-use disposable set comprising:
a housing configured to be inserted through a corresponding slot defined within an enclosure of a fluid injector wherein, upon installation of a multi-use disposable set within the fluid injector, a fluid connection port and a waste inlet port of the multi-use disposable set are accessible within the corresponding slot of the fluid injector;
a fluid inlet port defined through the housing, the fluid inlet port having a proximal side thereof being configured for connection to the fluid connection port of the multi-use disposable set;
a waste outlet port defined through the housing, the waste outlet port having a proximal side being configured for connection to the waste inlet port of the multi-use disposable set;
a patient fluid line having a fluid port connector, an end port connector and a flexible tubing extending between the fluid port connector and the end port connector, the fluid port connector being connected to a distal side of the fluid inlet port and the end port connector being removably connectable to a distal side of the waste outlet port;
such that (A) upon insertion of the housing into the corresponding slot of the fluid injector, the fluid inlet port and the waste outlet port connect, respectively, to the fluid connection port and the waste inlet port thereby enabling fluid communication between the multi-use disposable set and the single-use disposable set, and (B) upon removal of the housing from the corresponding slot, the fluid inlet port and the waste outlet port of the single-use disposable set disconnect, respectively, from the fluid connection port and the waste inlet port of the multi-use disposable set; and
a reconnection prevention mechanism that, upon removal of the housing from the corresponding slot, is configured to prevent reconnection of at least one of (A) the fluid inlet port of the single-use disposable set to the fluid connection port of the multi-use disposable set and (2) the waste outlet port of the single-use disposable set to the waste inlet port of the multi-use disposable set.

8. A single-use connector configured to be removably connected to a reusable connector, the single-use connector comprising:
a hollow, tubular housing having an open proximal side opposite a distal side along a longitudinal length of the housing; and
at least one guard member connected to the tubular housing configured to transition between an open position in which the single-use connector can be connected to the reusable connector and a closed position in which the at least one guard member prevents the single-use connector from being connected to the reusable connector,
wherein disconnecting the single-use connector from the reusable connector causes the at least one guard member to transition from the open position to the closed position.

9. The single-use connector of claim 8, further comprising at least one thread-less attachment member configured to be connected to a thread-less attachment member of the reusable connector to establish a fluid connection between the single-use connector and the reusable connector.

10. The single-use connector of claim 9, wherein the at least one thread-less attachment member of the single-use connector comprises at least one of a male luer connector and a female luer connector.

11. The single-use connector of claim 8, further comprising an o-ring provided around at least a portion of the housing for sealing the single-use connector to the reusable connector.

12. The single-use connector of claim 8, wherein the at least one guard member comprises an annular compression sleeve at least partially enclosing the open proximal end of the housing defining an interior and a proximal opening, wherein the distal end of the reusable connector can be inserted into the interior of the annular compression sleeve through the proximal opening and connected to the proximal end of the single-use connector when the at least one guard member is in the open position, and wherein, when the connectors are attached together, the at least one guard member engages at least a portion of the reusable connector to reinforce the connection between the single-use connector and the reusable connector.

13. The single-use connector of claim 12, wherein the annular compression sleeve comprises an elastomeric sleeve biased to the closed position, and wherein, when in the closed position, the sleeve at least partially covers the open proximal end of the housing to prevent reconnection of the single-use connector to the reusable connector.

14. The single-use connector of claim 12, wherein disconnecting the single-use connector from the reusable connector reduces a diameter of the opening defined by the annular compression sleeve, thereby preventing the single-use connector from being reconnected to the reusable connector.

15. The single-use connector of claim 12, wherein the annular compression sleeve is biased to the closed position.

16. The single-use connector of claim 12, wherein the annular compression sleeve comprises a distal portion mounted to and extending proximally and radially outwardly from the housing of the single-use connector, and a proximal portion connected to and extending proximally and radially inwardly from the distal portion of the annular compression sleeve, wherein the proximal portion of the compression sleeve defines the opening of the sleeve.

17. The single-use connector of claim 8, wherein the at least one guard member comprises at least a first arm and a second arm, wherein the first arm and the second arm each comprise a first portion connected to and extending proximally and radially outwardly from the housing of the single-use connector and a second portion connected to and extending proximally and radially inwardly from the first portion of the arm.

18. The single-use connector of claim 17, wherein, when the single-use connector is connected to the reusable connector, an inwardly directed surface of the second portion of each arm engages a portion of the reusable connector to reinforce a connection between the single-use connector and the reusable connector.

19. The single-use connector of claim 17, wherein disconnecting the single-use connector from the reusable connector causes the arms to pivot radially inwardly relative to the housing of the single-use connector, such that an outer surface of the second portion of the arms blocks reconnection of the single-use connector with the reusable connector.

20. The single-use connector of claim 8, wherein the at least one guard member is slidably positioned in the housing and wherein, when the single-use connector is in the closed position, a portion of the at least one guard member extends from the open proximal end of the housing to prevent the single-use connector from being reconnected to the reusable connector.

21. The single-use connector of claim 20, wherein the at least one guard member comprises a spring configured to bias the guard member to the closed position, a deployable support configured to transition from a retracted position to a deployed position, and at least one rod extending between the spring and the deployable support, and wherein a maximum outer diameter of the deployable support is greater when in the deployed position than when in the retracted position.

22. The single-use connector of claim 21, wherein the deployable support is maintained in the retracted position by a narrowed inner surface of a sidewall of the housing, and wherein the deployable support transitions to the deployed position once it extends beyond the open proximal end of the housing.
